(19) United States
(12) Patent Application Publication
(10) Pub. No.: US 2013/0122091 A1
(43) Pub. Date: May 16, 2013

(54) POLYDEOXYRIBONUCLEOTIDE-BASED COMPOSITIONS AND THEIR METHODS OF USE

(71) Applicants: Luisa Gennero, Torino (IT); Gainfranco Merizzi, Torino (IT)

(72) Inventors: Luisa Gennero, Torino (IT); Gainfranco Merizzi, Torino (IT)

(73) Assignee: MEDESTEA INTERNAZIONALE SPA, TOINO (IT)

(21) Appl. No.: 13/810,928

(22) PCT Filed: Jul. 20, 2011
(86) PCT No.: PCT/IB11/53240

§ 371 Date: Jan. 18, 2013

(30) Foreign Application Priority Data
Jul. 22, 2010 (IT) TO2010A000635

(51) Int. Cl. A61K 31/7088; A61K 31/59

(52) U.S. Cl. CPC A61K 31/7088; A61K 31/59
USPC 424/474; 424/43; 514/44 R

(57) ABSTRACT

Compositions including synergistic combinations of polydeoxyribonucleotides and methyl amides, such as carnitine, are provided. Such compositions may include further optional active ingredients, such as a calcium salt, vitamins of the D or B groups, a sodium salt and a potassium salt, preferably used in combination.

The present invention relates to a new therapeutic application for polydeoxyribonucleotides (hereinafter designated as “PDRNs”) and polydeoxyribonucleotide-based compositions suitable to be used in the new therapeutic application that forms the subject of the invention.

Therapeutic applications for polydeoxyribonucleotides are described in the prior art.

For instance, the International Patent Application WO2010/049898 describes the use of injectable PDRN compositions for the treatment of osteoarticular pathologies.

It is known that the cyanide-resistant respiration distinguishes the majority of plants and microorganisms from mammals and higher animals in general. Cyanide compounds are one of the poisons most frequently found in nature for defense against predators. Plants and microorganisms are provided with several elements that confer cyanide-resistance as well as an alternative cyanide-resistant respiratory chain. Such an alternative respiration generally relies on the presence of a unique protein, the alternative oxidase, designated as cyanide-insensitive Alternative OXidase (AOX), which delivers electrons directly from the quinone group of the mitochondrial respiratory chain to oxygen. Through AOX, the cytochrome portion of the respiratory chain is completely overcome, so as to strongly decrease the extrusion of protons bound to the oxidation substrate, at the same time lowering the production of ATP. The alternative AOX-dependent system is typical of plants and microorganisms, but not of mammals and higher animals in general. Conversely, in mammals and higher animals, a reduced redox state of the respiratory chain and a high level of pyruvate are the exact conditions arising from the hereditary diseases of human metabolism with mutations in the cytochrome C segment of the mitochondrial respiratory chain. Up to date, attempts to express AOX in human cells have failed.

There is an apparent need of new materials and methods for treating or alleviating the effects of a plurality of diseases and conditions related to deficiencies in the mitochondrial respiratory chain or, more in general, to genetic or acquired mitochondrial cytopathologies, effectively correcting all of the acidosis, metabolic acidosis and lactic acidosis conditions derived therefrom.

Therefore, one object of the present invention is to provide a composition effective in the therapeutic treatment and/or as a therapeutic adjuvant for acidosis conditions, such as in particular metabolic acidosis and/or lactic acidosis, including acidosis cases with serious vascular damage complicated by ischemic or thrombotic events for anti-acidosis, anti-aggregation, vasculature-protective, antioxidant, anti-bone demineralization, re-equilibration of calcium and fatty acid cell metabolism, re-equilibration of mitochondrial and cellular metabolism, reduction and neutralization of the gastroenteric lactate deposits and ultimately anti-apoptotic effects, both in vitro and in vivo, in the human and veterinary field, and thus suitable for pharmaceutical and cosmetic applications, or as a dietary supplement.

These and other objects are attained by the studies carried out by the present inventors, which found that polydeoxyribonucleotide (PDRN)-based compositions are effective in the treatment of acidosis conditions, as mentioned above.

Within the scope of the present description, the term polydeoxyribonucleotides (PDRNs) is meant to indicate a mixture of deoxyribonucleotide chains of different molecular weights, obtained from natural and/or synthetic sources, preferably of animal or plant origin, such as fish placenta or sperm or plants. Fish sperm is a preferred natural source. The mixture of deoxyribonucleotide chains has a molecular weight distribution preferably comprised between 20 kDalton and 2500 kDalton, more preferably between 70 kDalton and 240 kDalton. In a preferred embodiment, the mixture of deoxyribonucleotide chains has a purity of at least 85%, preferably above 95% and more preferably above 98%.

PDRNs as defined above are known and commercially available substances.

FR 2 676 926 describes pharmaceutical compositions containing highly polymerized polydeoxyribonucleotides obtained from fish sperm, the compositions being used for the treatment or prevention of immune deficiencies. Compositions containing polydeoxyribonucleotides in a non-ionic solvent are described, which are usable by the parenteral route, particularly by intramuscular and/or intravenous route.

Methods for the preparation of polydeoxyribonucleotides from mammal placenta are described for example in EP 0 226 254.

Several biological activities and medical applications for PDRNs are also described in the prior art, the which are listed hereinafter.

PDRNs are known to trigger a metabolic stimulation of fibroblasts, which results both in an increase in the number of fibroblasts themselves (35%) and in an increase in the production of all the dermal matrix components (30%), that is collagen and non-collagen proteins, hyaluronic acid, elastin, fibronectin, etc. Such a PDRN-mediated secretion stimulates the purine receptors or cellular metabolic activation switches [1].

PDRNs are also known to activate the nucleic acid rescue routes (through salvage), reusing preformed nucleotides for cell duplication [2].

U.S. Pat. No. 3,829,567 describes the use of an alkaline metal salt of a ribonucleic acid (RNA) and/or deoxyribonucleic acid (DNA) polynucleotide or oligonucleotide as a fibrinolytic drug.

U.S. Pat. No. 4,649,134 describes a method of treatment for acute kidney failures accompanied by thrombotic microangiopathy with PDRNs; such pathologies include hemolytic uremic syndrome (HUS), collagen disorders (for example, panarteritis and lupus), Wegner, Schoenlein-Henoch, disseminated intravascular coagulation (DIC), rapidly progressive glomerulonephritis, thrombocytopenia and thrombotic purpura (TPP).

U.S. Pat. No. 4,693,995 aims at elucidating the use of PDRNs in the treatment of acute conditions of myocardial ischemia and heart attack.

PDRNs are also known to increase the production and the occurrence of VEGF (endothelial growth factor) in skin and mucosae [3]. This results in an increase in local vascularization, with a consequent increase in the circulatory flow and the district oxygenation.

PDRNs are also known to induce the release of the plasminogen activator from the vascular tissue and decrease the blood concentration of plasmin inhibitors.

Further pharmacological investigations showed that PDRNs affect arachidonic acid metabolism within the vessel wall, causing an increase in the prostacyclin (PGI-2) availability. This endothelium-produced substance is the most active inhibitor of platelet clustering known so far, as well as an effective vasodilator. The remarkable anti-thrombotic and pro-fibrinolytic activities of the PDRNs derive from these pharmacological effects.

PDRNs were also found to have a synergistic action and they enhance the effect of heparin [4]. The proposed synergistic mechanism of PDRNs and heparin is that the PDRNs competitively bind to heparin receptors, promoting a sustained circulation of endogenous heparin. Cetingli A I et al. pointed out an increase in the anti-factor Xa activity, suggesting an effective anti-thrombotic action [4].

Treatment with PDRNs was described to be able to induce inhibition of PAF (Platelet Activating Factor)-mediated superoxide production by neutrophils, demonstrating a protective effect in mice against pulmonary embolism [5].

PDRNs can also induce mobilization of Peripheral Blood Progenitor Cells (PBPC) in a murine model [6]. Certain PDRNs in synergism with growth factor rhG-CSF (Recombinant Human Granulocyte Colony-Stimulating Factor) significantly increase the mobilization of many PBPCs, comprising the early progenitor cells (HSCs, Hematopoietic Stem Cells). These data may have considerable implications for anti-tumor therapy in human beings.

The hepatic veno-occlusive disease is the most common of the toxicities related to the accompanying regime for stem cell transplant. In spite of the aggressive therapies, comprising the combination of tissue plasminogen activator and heparin, the serious hepatic veno-occlusive disease is nearly always lethal. PDRNs act in many vascular disorders and, unlike plasminogen and heparin, do not produce any systemic anticoagulant effect [6]. Resolution of the serious hepatic veno-occlusive disease was observed in 42% of PDRN-treated survivors, versus 2% of non-PDRN-treated survivors. The observed response rate and the absence of toxicity of the treatment in connection with PDRNs are convincing for an effective therapeutic effect in case of hepatic veno-occlusive disease [6-7].

However, as far as the present inventors know, the effectiveness of PDRNs in the treatment of simple or complicated, acute or chronic acidosis conditions, has not yet been described.

The inventors recognize that the use of PDRNs is described in the state of the art for treating ischemic conditions. For instance, Bitto Alessandra et al., Journal of Vascular Surgery, vol. 48, no. 5, November 2008, pages 1292-1300, shows that PDRNs are effective in restoring the blood flow in an experimental model of peripheral artery occlusive disease (hind leg ischemia), by stimulating the production of vascular endothelial growth factor (VEGF).

The inventors also recognize that ischemia in some cases, but not in all cases, may represent a secondary complication to lactic or metabolic acidosis conditions.

However, the induced ischemic state in the above-mentioned reference Bitto et al., 2008, has no connection with lactic or metabolic acidosis. Moreover, the stimulation of VEGF production described in Bitto et al., 2008 does not represent a suggestion for the person skilled in the art of the possible effectiveness of PDRNs in the treatment of lactic or metabolic acidosis conditions, as the effectiveness in the treatment of lactic or metabolic acidosis relies on activation of completely different mechanisms, which are schematized herein-below:

I. PDRNs assist in the activation of the organic buffer systems (in vitro, following administration of PDRN-containing solutions, a blockage of the pH shift towards the low range occurs, that is a blockage of the worsening of the immediate acidosis condition).
II. PDRNs, in an acidic environment, prevent the binding between receptors and adhesion molecules, playing a role in suppressing the reactive inflammatory conditions due to change of the pH towards acidosis.
III. PDRNs, in an acidic environment, favor recaptation.
IV. PDRNs, in an acidic environment, contribute to maintaining the proper viscosity of extracellular liquids and the proper osmotic pressure.
V. PDRNs, in an acidic environment, contribute to stabilizing the expression of trans-membrane receptor glucose transporters, occurring in the majority of mammal cells. The action thereof allows for glucose transport across plasma membranes. The most known and studied glucose transporter is GLUT-4, because of its direct insulin sensitivity. Under normal conditions, this carrier is found in the cytoplasm and the translocation thereof onto the cell membrane is stimulated by insulin binding to the membrane receptor. This process favors glucose displacement from the interstitial liquid to the interior of the cell. When blood glucose concentration normalizes and insulin is cleared, GLUT 4 molecules are slowly removed from the plasma membrane and sequestered by endocytosis into intracellular vesicles. During pH imbalances, glucose transport across cell membranes may be accelerated or compromised. During acidosis, the cells inefficiently use an enormous amount of glucose in order to survive. This way they produce lactate as a waste product, promoting transition from metabolic acidosis to lactic acidosis. PDRNs appear to correct glucose consumption at the cellular level, inhibiting the expression of membrane receptors and favoring the district oxygenation, resulting in a clear increase in cell and tissue viability in an acidic environment (in vitro data).

Altogether, activation of the above-schematized mechanisms defines the containment of the ion imbalance, which is typical of metabolic acidosis (characterized by a decrease in the concentration of HCO₃) and lactic acidosis (characterized by a build-up of lactic acid in the body, with a consequent decrease in lactic acid catabolism and acidification of blood) conditions.

Other prior art references have described the effectiveness of defibrotide in hindering the effects of ischemic conditions at the cardiac (Niada R et al., Haemostasis 1986, vol. 16, suppl. 1, pages 18-25) and hepatic (Ferrero M et al., Journal of Hepatology, vol. 10, no. 2, 1990, pages 223-227) levels. These references clearly indicate that defibrotide acts by causing an increase in the production of prostacyclin (PGI₂). The same mechanism is described in Ferrero M et al., Journal of Tissue Reactions, vol. 11, no. 4, 1989, pages 179-184, and in Rossoni G et al., Journal of Cardiac Surgery, September-October 1999, vol. 14, no. 5, pages 334-341.

A first object of the invention thus concerns the use of polydeoxyribonucleotides (PDRNs) as a medicament for the treatment of acidosis conditions, particularly lactic and/or metabolic acidosis, as defined in the appended claims.

A second object of the invention is a composition comprising the combination of polydeoxyribonucleotides (PDRNs) and at least one methyl amide, preferably carnitine, for use in the treatment of acidosis conditions, particularly lactic and/or metabolic acidosis, as defined in the appended claims.

The appended claims are an integral part of the technical teachings of the present description.

As will be described in detail in the following experimental section, the polydeoxyribonucleotides (PDRNs) and the polydeoxyribonucleotide (PDRN)-based compositions of the present invention proved to be effective in counteracting the genetic or acquired defects in mitochondrial oxidative phosphorylation and in effectively correcting acidosis conditions, such as metabolic acidosis, lactic acidosis and the consequent cell apoptosis. PDRNs in combination with at least one methyl amide, preferably carnitine, also showed a synergistic effect.

The inventors demonstrated that the PDRNs and compositions of the invention are capable of counteracting acidosis conditions in general and, by acting on more than one level, of restoring the optimum physiological and biochemical conditions to protect the viability and trophism of the tissues involved and protecting from ischemic or thrombotic degenerative conditions.

Experiments carried out in vitro demonstrated that the PDRNs and compositions of the invention have significant anti-aggregation, antioxidant, anti-apoptotic and anti-lactacidemic activities, with consequent anti-decalcifying and anti-osteoporotic effects.

As the regenerative and restoring properties detected in vitro are also maintained in vivo, the PDRNs and compositions of the invention lend themselves to many in vivo applications, such as medicaments or medical devices or dietary supplements with anti-acidosis, anti-ischemic, anti-aggregation and anti-osteoporotic activities, particularly for treatment of pathologies or medical conditions both in human beings and in animals, such as genetic or acquired defects in the mitochondrial oxidative phosphorylation and acidosis conditions in general, such as metabolic acidosis, lactic acidosis, acidosis-dependent hypocalcemia, acidosis-dependent osteomalacia and, more generally, acidosis-dependent reactive fibrosis and cell apoptosis following acidosis conditions.

As will be described in more detail hereinbelow, the compositions of the invention comprise the combination of PDRNs as defined above and at least one methyl amide, preferably selected from the group consisting of carnitine, a carnitine ester, such as preferably acetylcarnitine or propionylcarnitine, acylcarnitine, levorotatory forms and simple or complexed derivatives thereof.

In a preferred embodiment, the compositions of the invention comprise further ingredients that assist in the effectiveness of the compositions; such further ingredients are preferably selected from the group consisting of calcium salts, vitamins of the D or B groups, potassium salts together with sodium salts, and combinations thereof.

Among the calcium salts suitable for use in the compositions of the invention, calcium citrate, monocalcium citrate, tricalcium citrate, calcium carbonate, dibasic calcium phosphate, calcium dicitrate, tricalcium dicitrate tetrahydrate, calcium chloride, calcium gluconate, calcium phosphate, calcium nitrate are mentioned by way of example.

Among the vitamins suitable for use in the compositions of the invention, vitamins of the D group such as calciferol, ergocalciferol and lumisterol, cholecalciferol, dihydroergocalciferol, sitocalciferol, calcitriol, calcifediol; vitamins of the B group such as thiamine (vitamin B1) and/or riboflavin (vitamin B2) and/or niacin, nicotinic acid or nicotinamide (vitamin B3) and/or adenine (vitamin B4 or vitamin-like factor) and/or pantothenic acid, pantenol, pantethine (vitamin B5) and/or pyridoxamine, pyridoxine and pyridoxal (vitamin B6); and derivatives thereof are mentioned by way of example.

Sodium salts and potassium salts are preferably used in a combination, such as for example the following combinations: sodium citrate and/or potassium citrate, monosodium citrate and/or monopotassium citrate, disodium citrate, trisodium citrate and/or tripotassium citrate, sodium carbonate and/or potassium carbonate, sodium bicarbonate and/or potassium bicarbonate, sodium sulfate and/or potassium sulfate, sodium bisulfate and/or potassium bisulfate, sodium chloride and/or potassium chloride, sodium tartrate and/or potassium tartrate, sodium phosphate and/or potassium phosphate, disodium inosinate and/or dipotassium inosinate, sodium ascorbate and/or potassium ascorbate, sodium nitrate and/or potassium nitrate.

Mitochondrial toxicity is a condition wherein mitochondria are damaged by an acute or chronic toxicity condition of cells or tissues, or they undergo a restorable or irreparable sudden oxidation, or they suffer a significant decrease in the total number of cell, tissue or body organelles. The exact causes of mitochondrial toxicity are unknown. The cell function disorder that goes with the mitochondrial toxicity condition may initially cause mild disturbances such as muscle weakness and myopathies in general, then disorders such as peripheral neuropathies and pancreatitis, up to very serious problems such as acute or chronic acidosis conditions and in particular metabolic acidosis and lactic acidosis. Buildup of lactic acid in the body tissues results in loss of energy and organ function, and ultimately death.

PDRNs extracted from natural and/or synthetic sources, and the PDRN-based compositions of the present invention proved to be fully effective in the treatment of pre-acidosis and acidosis conditions, particularly of metabolic and lactic acidosis conditions, including acidosis cases with serious vessel damage complicated by ischemic or thrombotic events, and all this thanks to the anti-acidosis, anti-aggregation, vasculature-protective, anti-apoptotic, anti-demineralization, antioxidant effects and the decrease, containment and neutralization of lactate build-up at the gastroenteric level.

PDRNs extracted from natural and/or synthetic sources, and the PDRN-based compositions of the present invention can be used in any suitable pharmaceutical dosage form, for example as an oral gel, oral solution, tablets, solutions for rectal administration, solid substances for rectal administration, lyophilized powder substances, or injectable solutions.

As mentioned above, the PDRNs used in the present invention are obtained from a DNA-rich animal or plant source by methods described in the above-mentioned literature. The selection of fish sperm as the PDRN source is preferred.

The novel use of the PDRNs, which forms the subject of the invention, is based on experimental pharmacology studies performed in vitro and in vivo by the present inventors who showed that the PDRNs have an important anti-acidosis effect on tumor cell lines, mammal primary cell cultures, and mammal tissue biopsies.

The in vitro experimental findings appear consistent with the in vivo clinical observations performed on mammals.

The in vitro and in vivo experimental findings are described in detail below.

As previously mentioned, PDRNs are used as such or in combination with other active ingredients, which have been selected for their enhancing and synergistic activity with PDRNs, according to the type of acidosis condition to be treated and complications thereof.

The combination with carnitine or derivatives thereof synergistically enhances the anti-acidosis, anti-aggregation, vasculature-protective, and antioxidant effects of the PDRNs, because of their limiting and counteracting action on the ongoing mitochondrial damage during the acute or chronic acidosis condition. The synergistic effects are even more evident when the metabolic or lactic acidosis conditions are symptomatic and, particularly, when they are worsened by myopathy and myasthenia gravis.

Calcium salts serve two functions that are synergistic with one another, that is: (i) correction of dysemia (imbalance of mineral salts in blood), which arises as the first tissue outcome from the acidosis condition; (ii) buffering action on bone, which generally occurs several hours after induction of acidosis and, as for metabolic acidosis, during the chronicization phase. It is known that bone is made up of both an organic part and an inorganic part. The latter, which represents about ⅔ by weight of the mature bone, contains hydroxyapatite crystals [Ca₁₀(PO₄)₆(OH)₂]. The organic portion, designated as osteoid, mainly consists of type I collagen and cells, osteoblasts and osteoclasts, which assist in the remodeling process. Such a remodeling, as known for example from WO 2009115602 and [8], is induced by PDRNs. In case of acidosis, whether acute or chronic, of metabolic or respiratory origin, changes in the chemical composition of the bone occur, especially in the non-osteoid inorganic portion [9]. Despite the fact that respiratory acidosis has less serious effects on bone metabolism than metabolic acidosis, a few studies [10-11] demonstrated that acidosis, however, is able to inhibit osteoblast activity and stimulate osteoclast activity in vitro, with a consequent and progressive loss of calcium and sodium by the bone and an increase in H⁺ entry. In addition, a decrease in the amount of carbonate is observed, which may, in the long term, lead to a serious deficiency. In vivo, the loss of CaCO₃ by the bone has a crucial role in the onset of osteomalacia both in CRF patients and in patients with fully functioning kidneys. PDRNs, whenever in association with calcium ions or salts or derivatives thereof, synergistically enhance the anti-acidosis, anti-bone demineralization effect, restoring a physiological balance between the osteoblast and osteoclast components and especially compensating for the loss of the inorganic non-osteoid portion. The synergistic effects are even more evident when the metabolic or lactic acidosis conditions are symptomatic and, particularly, when they are worsened by true osteomalacia, or hypocalcemia or hypercalcemia and/or hypocalciuria or hypercalciuria related to acute or chronic acidosis conditions, secondary hyperprolactinemia or secondary hyperparathyroidism.

Vitamins of the D group, in synergistic combination with PDRNs, become co-factors that enhance the metabolizing action during the phases of calcium absorption, with osteo-regenerating effects during treatment of serious symptomatic metabolic or lactic acidosis, especially when worsened by osteomalacia and myasthenia.

Vitamins of the B group, in synergistic combination with PDRNs, become co-factors that enhance the endothelium-protection, antioxidant and neuro-regeneration actions during treatment of serious symptomatic metabolic or lactic acidosis, especially when worsened by peripheral neuropathy.

PDRNs in combination with a sodium salt together with a potassium salt, preferably sodium citrate and/or potassium citrate, in vivo have a synergistic and therapeutic action of decreasing, containing, and neutralizing lactate build-up at the gastroenteric level and in vitro they induce an anti-apoptotic/anti-cytopathic effect, in the treatment of acidosis conditions, metabolic acidosis, lactic acidosis, particularly during serious chronic or acute, simple or complicated acidosis.

The compositions of the invention exhibit a regeneration activity on cells and tissues, such as to allow, in vitro, for the maintenance of primary cells or cell lines and tissue biopsies in culture under optimum viable conditions, inducing the regeneration thereof.

The histological results obtained in vitro after treatment with the compositions of the invention confirm the induction of regeneration and re-trophization of cells and tissues, which appear morphologically comparable to in vivo intact tissues and with optimum his-to-functional characteristics (see Example 1).

In vitro treatment with the compositions of the invention of biopsy tissues damaged by a high acidity of the culture medium results in a surprising repairing of the damages and a total recovery of the original trophism. All the tissues depicted in the treated biopsy samples undergo a complete, functional and morphological regeneration, consequent to a possible absence or reduced presence of degeneration processes.

The hematochemical results (hemochrome, venous EGA, lactacidemia, calcemia, alkaline phosphatase, LDH, CPK, INR) obtained in vivo at time point zero versus time points 7 days, 15 days, 30 days, of treatment with the compositions that form the subject of the present invention and combined formulations thereof (see the section related to clinical studies), confirm an induction to an optimum functional recovery or a complete functional recovery, depending on the seriousness of the acidosis condition at the beginning of the treatment.

The following tables, which are provided solely for illustration and not limitation, represent specific embodiments of the compositions of the invention, each specifically designed for a particular type of application that promotes a functional recovery during treatment of acute or chronic acidosis conditions.

The compositions provided in the tables may be modified both in quantity and ingredients, according to what indicated in the claims, yet remaining within the scope of the invention.

Without wishing to be bound by any relevant specific theory, the present inventors believe that the results obtained with the present invention demonstrate that the acidosis condition is recoverable. Acidosis in general, whether acute or chronic, metabolic, lactic, early or worsened by serious degenerative dysfunctions, proved to be treatable with the PDRNs and compositions that form the subject of the invention.

### COMPOSITION EXAMPLES

### 1) PDNR Formulation for Oral Administration

Anti-acidosis composition with vasculature-protective, anti-aggregation, anti-ischemic, anti-thrombotic, osteo-protective, antioxidant effects.

| Substance | Concentration |
|---|---|
| PDNR | 100 mg/g |
| Excipients | q.s. to 1 g of final composition |

### 1-bis) PDNR Formulation for Cell and Tissue Cultures

Anti-acidosis composition with antioxidant and anti-apoptotic effects.

| Substance | Concentration |
|---|---|
| PDNR | 5 mg/500 ml |
| Complete culture medium | q.s. to 500 ml of solution |

### 2) Formulation of PDNR and a Methyl Amide, for Oral Administration

Anti-acidosis composition with antioxidant, anti-myasthenic, anti-myopathic effects.

| Substance | Concentration |
|---|---|
| PDNR | 100 mg/g |
| L-carnitine | 424 mg/g |
| Excipients | q.s. to 1 g of final composition |

### 2-bis) Formulation of PDNR and a Methyl Amide, for Cell and Tissue Cultures

Anti-acidosis composition with antioxidant and eutrophizing effects.

| Substance | Concentration |
|---|---|
| PDNR | 5 mg/500 ml |
| L-carnitine | 25 mg/500 ml |
| Complete culture medium | q.s. to 500 ml of solution |

### 3) Formulation of PDNR, Methyl Amide and a Calcium Salt for Oral Administration

Anti-acidosis composition with anti-demineralization, anti-osteomalacia effects.

| Substance | Concentration |
|---|---|
| PDNR | 100 mg/g |
| L-carnitine | 424 mg/g |
| Calcium carbonate | 150 mg/g |
| Excipients | q.s. to 1 g of final composition |

### 3-bis) Formulation of PDNR, Methyl Amide and a Calcium Salt, for Cell and Tissue Cultures

Anti-acidosis composition with a calcium re-equilibration effect

| Substance | Concentration |
|---|---|
| PDNR | 5 mg/500 ml |
| L-carnitine | 25 mg/500 ml |
| Calcium carbonate | 20 mg/500 ml |
| Complete culture medium | q.s. to 500 ml of solution |

### 4) Formulation of PDNR, Methyl Amide and a Vitamin of the D Group for Oral Administration

Anti-acidosis composition with anti-demineralization, anti-osteoporosis effects.

| Substance | Concentration |
|---|---|
| PDNR | 100 mg/g |
| L-carnitine | 424 mg/g |
| Calcitriol | 0.5 μg/g |
| Excipients | q.s. to 1 g of final composition |

### 4-bis) Formulation of PDNR, Methyl Amide and a Vitamin of the D Group, for Cell and Tissue cultures

Anti-acidosis composition with a calcium malabsorption correction effect.

| Substance | Concentration |
|---|---|
| PDNR | 5 mg/500 ml |
| L-carnitine | 25 mg/500 ml |
| Calcitriol | 0.25 μg/500 ml |
| Complete culture medium | q.s. to 500 ml of solution |

### 5) Formulation of PDNR, Methyl Amide and a Vitamin of the B Group for Oral Administration

Anti-acidosis composition with antioxidant and anti-neuropathic effects.

| Substance | Concentration |
|---|---|
| PDNR | 100 mg/g |
| L-carnitine | 424 mg/g |
| Ribloflavin | 100 mg/g |
| (vitamin B2) | |
| Excipients | q.s. to 1 g of final composition |

### 5-bis) Formulation of PDNR, Methyl Amide and a Vitamin of the B Group, for Cell and Tissue cultures

Anti-acidosis composition with antioxidant and pro-differentiation effects.

| Substance | Concentration |
|---|---|
| PDNR | 5 mg/500 ml |
| L-carnitine | 25 mg/500 ml |
| ribloflavin | 10 mg/500 ml |
| (vitamin B2) | |
| Complete culture medium | q.s. to 500 ml of solution |

### 6) Formulation of PDNR, Methyl Amide and a Sodium Salt Together with a Potassium Salt for Oral Administration

Anti-acidosis composition with antioxidant and anti-lactacidemic effects.

| Substance | Concentration |
|---|---|
| PDNR | 100 mg/g |
| L-carnitine | 424 mg/g |
| sodium citrate | 200 mg/g |
| potassium citrate | 25 mg/g |
| Excipients | q.s. to 1 g of final composition |

### 6-bis) Formulation of PDNR, Methyl Amide and a Sodium Salt Together with a Potassium Salt, for Cell and Tissue Cultures

Anti-acidosis composition with antioxidant and anti-lactacidemic effects.

| Substance | Concentration |
|---|---|
| PDNR | 5 mg/500 ml |
| L-carnitine | 25 mg/500 ml |
| sodium citrate | 20 mg/500 ml |
| potassium citrate | 2.5 mg/500 ml |
| Complete culture medium | q.s. to 500 ml of solution |

### 7) Formulation of PDNR, Methyl Amide, a Calcium Salt, a Vitamin of the D Group, a Vitamin of the B Group, a Sodium Salt Together with a Potassium Salt, Administration by Oral Route

Composition for acute- or chronic-phase acidosis conditions, with a high lactacidemia.

| Substance | Concentration |
|---|---|
| PDNR | 100 mg/g |
| L-carnitine | 424 mg/g |
| Calcium carbonate | 150 mg/g |
| Calcitriol | 0.5 μg/g |
| ribloflavin | 100 mg/g |
| (vitamin B2) | |
| sodium citrate | 200 mg/g |
| potassium citrate | 25 mg/g |
| Excipients | q.s. to 1 g of final composition |

### 7-bis) Formulation of PDNR, Methyl Amide, a Calcium Salt, a Vitamin of the D Group, a Vitamin of the B Group, a Sodium Salt Together with a Potassium Salt, for Cell and Tissue Cultures

Composition for serious acidosis conditions with a high lactacidemia.

| Substance | Concentration |
|---|---|
| PDNR | 5 mg/500 ml |
| L-carnitine | 50 mg/500 ml |
| Calcium carbonate | 20 mg/500 ml |
| Calcitriol | 0.25 μ g/500 ml |
| ribloflavin (vitamin B2) | 10 mg/500 ml |
| sodium citrate | 20 mg/500 ml |
| potassium citrate | 2.5 mg/500 ml |
| Complete culture medium | q.s. to 500 ml of solution |

It will be understood that the formulations specifically illustrated in the tables above are non-limiting examples of specific embodiments of the present invention.

In the context of the present invention, PDRNs, optionally in combination with a methyl amide and the additional optional ingredients mentioned previously, may be effectively employed in a wide range of concentrations, as defined in the appended claims.

The PDRNs and compositions of the invention may be formulated into solid or liquid formulations, for in vivo or in vitro use.

In vivo use is intended to mean the use as a drug, a medical device or a dietary supplement.

Solid formulations for in vivo use are, for example, tablets, lozenges, suppositories, powders, granules, etc.; they comprise, in addition to the active principle (or active principles), conventional physiologically and/or pharmaceutically acceptable excipients, the selection and use of which is well within the skills of the person of ordinary skill in the art.

Liquid formulations for in vivo use are, for example, solutions or suspensions to be administered by injection; besides the above-described active principle (or active principles), they comprise conventional physiologically and/or pharmaceutically acceptable solvents, such as for example isotonic saline solutions.

Solid and liquid formulations for in vivo use are generally administered from 1 to 5 times daily depending on the seriousness of the acidosis condition to be treated.

Formulations for in vitro use are generally liquid and are usually formulated as cell and/or tissue culture media; in addition to the physiologically acceptable liquid medium, they also comprise the usual components of in vitro culture media for eukaryotic cells or tissues, such as for example amino acids, sugars, salts, vitamins, etc.

### Composition Rationale

The present invention is based on the observation of a particular anti-acidosis action performed by PDRNs extracted from natural and/or synthetic sources, alone or in combination with methyl amide and optionally a calcium salt, a vitamin, preferably of the D or B group or derivatives thereof, and a sodium salt together with a potassium salt, preferably sodium and/or potassium citrate.

PolyDeoxyRiboNucleotides (PDRNs), derived from nucleic acid degradation, are extremely well tolerated molecules that enter the physiological catabolism of nucleic acids. Polynucleotides are known to exert a physiological stimulus to cell proliferation and tissue repair in damaged tissues. It is also known that by-products from the enzymatic degradation of polynucleotide chains (simple nucleotides, nucleosides, nitrogenous bases) occur physiologically in the extracellular milieu and are useful trophic substrates for promoting cell regeneration and metabolic activity. In vivo, polynucleotides and nucleotides are used at the tissue level both to improve cell activity and to protect and promote physiological repair and regeneration mechanisms.

Methyl amides, and in particular the most known activities of carnitine and salts and esters thereof are mitochondrial beta-oxidation of long-chain fatty acids (from the biochemical point of view, carnitine exerts its functions by participating in a complex mechanism termed carnitine acyl-CoA transferase) and regulation of glucose use. Carnitine and esters thereof operate by causing stabilization of cell and mitochondria membranes, which is essential for cell repair processes and the functionality of the cell itself. Further endogenous aggravating factors are added to the pro-acidosis degenerative processes, such as the generation of oxygen free radicals. The optimum ability of cell reaction to noxious stimuli goes through the maintenance of energy production and osmotic balance. Carnitine is involved in the intermediate metabolism of lipids and carbohydrates, which is essential for cell function.

The methyl amide is selected from the group consisting of carnitine, L-carnitine, acetylcarnitine, L-acetylcarnitine, acylcarnitine, levocarnitine, or derivatives thereof.

Calcium ions and calcium salts contribute to the normalization of the loco-regional microenvironment, by promoting an ideal sodium-calcium ion exchange and favoring the correction of the modified electrolyte concentration to physiological values: they play an auxiliary role in the consolidation of osmosis, viscosity, and pH of the extracellular microenvironment where cutis and subcutis occur. Moreover, by restoring a physiological microenvironment, it is possible to promote activation of two repair mechanism in the organism: attraction of stem cells from the circulatory system and increased specialization of skin-resident stem cells towards a fast differentiation into mature cells designed to restore damaged tissues. During metabolic acidosis, a progressive loss of calcium and sodium occurs in the bone with an increase in the entrance of H+ and a decrease in the amount of carbonate, which may, in the long term, lead to a serious deficiency. The loss of CaCO₃ from the bone plays a critical role in the onset of osteomalacia both in patients with chronic renal failure and in those with a maintained renal function. The importance of restoring the physiological calcium compartment appears evident during acute and chronic acidosis.

The calcium salt is selected from the group consisting of calcium citrate, monocalcium citrate, tricalcium citrate, calcium carbonate, dibasic calcium phosphate, calcium dicitrate, tricalcium dicitrate tetrahydrate, calcium chloride, calcium gluconate, calcium phosphate, calcium nitrate, or derivatives thereof.

Vitamins of the D and B groups enhance a correct tissue nutrition, bringing about the gradual physiological restoration of the physiological microenvironment (restoration of the correct pH and antioxidant potential, correction of electrolyte absorption). In addition, more generally, the vitamins in the organism exert many biological functions related to the complex cell differentiation process.

The vitamin of the D group is selected from the group consisting of calciferol, ergocalciferol and lumisterol, cholecalciferol, dihydroergocalciferol, sitocalciferol, calcitriol, calcifediol, or derivatives thereof.

The vitamin of the B group is preferably selected from the group consisting of thiamine (vitamin B1) and/or riboflavin (vitamin B2) and/or niacin, nicotinic acid or nicotinamide (vitamin B3) and/or adenine (vitamin B4 or vitamin-like factor) and/or pantothenic acid, pantenol, pantenine (vitamin B5) and/or pyridoxamine, pyridoxine and pyridoxal (vitamin B6), or derivatives thereof.

The deficiency in our diet of basic components (occurring as potassium and magnesium organic salts in vegetables, which were abundantly eaten by our ancestors) and the replacement thereof with salt (sodium chloride, almost absent in vegetables and used in huge quantities in our current diet) does not allow to neutralize the “acid load” produced by acidogenic food, that is food that produces acids derived from sulphur, phosphorous and chloride metabolisms in the organism. Thereby, a “chronic latent metabolic acidosis” condition develops, which tends to worsen with ageing, as a consequence of the physiological decline of the renal function (critical for maintaining the organism's acid-base balance). Acid-base balance is extremely important for protein structure and function, cell membrane permeability, electrolyte distribution. Organisms have several systems for adjusting such a balance, based on the buffering abilities of blood and intra- and extra-cellular liquids, gas exchange at the lung level, and renal secretion. Think of blood: its pH varies within a very narrow range (7.37 and 7.43); in order to keep these values constant, bicarbonate is the first to intervene, followed by hemoglobin, plasma proteins, and phosphate. This combination of buffers is very efficient and allows for a rapid and continuous blood pH adjustment. Carbon dioxide clearance from lungs allows to avoid a decrease in blood pH; the same result is attained with secretion into urine of protons (H) derived from degradation of several metabolites. Organic salts (such as citrate) of minerals and oligoelements are particularly important for an effective proton neutralization. These salts dissociate releasing organic anions (citrate). Anions—in connection with the dissociation constant of the corresponding acids—associate with protons, giving rise to acids, which subsequently are transformed into carbon dioxide and water. Instead, the cation is reabsorbed at the renal level (exchange with other protons), which results in a further elimination of acidity.

A sodium salt together with a potassium salt is selected from the group consisting of sodium citrate and/or potassium citrate, monosodium citrate and/or monopotassium citrate, disodium citrate, trisodium citrate and/or tripotassium citrate, sodium carbonate and/or potassium carbonate, sodium bicarbonate and/or potassium bicarbonate, sodium sulfate and/or potassium sulfate, sodium bisulfate and/or potassium bisulfate, sodium chloride and/or potassium chloride, sodium tartrate and/or potassium tartrate, sodium phosphate and/or potassium phosphate, disodium inosinate and/or dipotassium inosinate, sodium ascorbate and/or potassium ascorbate, sodium nitrate and/or potassium nitrate, or derivatives thereof.

The cosmetic, pharmaceutical, dietary supplement, medical device, and tissue culture compositions, which form the subject of the present invention, may also comprise further accessory elements such as excipients and carriers, the selection and use of which is well within the skills of the person of ordinary skill in the art without the need of the exercise of any inventive skill.

The culture media subject of the invention may also comprise further ingredients, such as for example the usual inorganic salts, sugars, peptides, amino acids, and vitamins required for culture maintenance and/or growth of mammal cells, as well as optional antibiotic and/or antimicrobial agents required to avoid contamination of the cultures.

Among the amino acids that may be present in the compositions of the invention, we mention, by way of example, methionine, cystine, N-acetylcysteine, cysteine, glycine, leucine, isoleucine, proline, glutamine, arginine, glutamic acid, histidine, histidine-HCl, lisine, lisine-HCl, phenylalanine, serine, threonine, tryptophan, tyrosine, tyrosine disodium salt, valine, proline, hydroxyproline. Such amino acids are often used in mixtures comprising a high number of different amino acids. Besides the amino acids, the compositions of the invention nay also comprise peptides and proteins, such as glutathione, collagen, elastin, wheat extract, and the like.

Examples of cell culture solutions are for example RPMI 1640 (cell culture medium), DMEM-LG (cell culture medium), AIM-V (cell culture medium), high-glucose modified D-MEM (cell culture medium), EBM (cell culture medium), human albumin, FBS (fetal bovine serum for cell cultures), F12 (cell culture solution containing a complete amino acid source), HANK's solution (cell culture solution containing sodium bicarbonate).

Finally, within the antibiotic and antimicrobial category, we mention, by way of example, gentamicin, penicillin, streptomycin, ciprofloxacin, levofloxacin, metronidazole, chlorhexidine, amphotericin B, fluconazole, itraconazole, triazole antimycotics, silver (it has a bacteriostatic activity).

### Example 1

### Cell Cultures, Biopsies and Prototypic Solutions

### Cell Cultures Under Examination

The test biopsy samples are listed below:
Monocyte cell line THP-1;
Canine primary hepatic cells;

### Biopsies

The test biopsy samples are listed below:
canine hepatic biopsies;
ovine stromal biopsies.

All the samples were washed three times with physiological solution and antibiotics (100 units/ml penicillin+100 μg/ml streptomycin+40 mg/L gentamicin, fluconazole 0.2 mg/ml) for 10 min at room temperature.

The cells were divided into nine aliquots (two controls and seven samples to be treated for each patient) and each suspended at a concentration of 250×10³ cells/ml in a control solution (tested in duplicate) or in solutions 1-bis, 2-bis, 3-bis, 4-bis, 5-bis, 6-bis and 7-bis, in 24-well plates (Lab-Tek chamber slides, Nunc, Kamstrup, Denmark) in a final volume of 2 ml/well, to allow for a physiological perspiration.

The biopsies were then sectioned into nine parts (two controls and seven samples to be treated for each patient) and each suspended in 4 ml of a control solution (tested in duplicate) or in solutions 1-bis, 2-bis, 3-bis, 4-bis, 5-bis, 6-bis and 7-bis, in 15 cm-diameter wells (Lab-Tek chamber slides, Nunc, Kamstrup, Denmark), to allow for a physiological perspiration.

1. The control samples were then treated with D-MEM/F12 medium supplemented with:

### 10% FBS (Celbio, Milan, Italy)

100 units/ml penicillin
100 μg/ml streptomycin
160 mg/L gentamicin (Schering-Plough, Milan, Italy)
0.2 mg/ml fluconazole (Pfizer Italia S.r.l.),
2 mM L-glutamine (Life Technologies; growth medium).

The conditions as described in Tables 1-bis, 2-bis, 3-bis, 4-bis, 5-bis, 6-bis and 7-bis were added to the control culture medium. Throughout the experimental incubation no addition or replacement of fresh culture medium was done.

All the samples were placed into a Heraeus incubator thermostatically controlled at the temperature of 37° C. with an atmosphere containing a continuous input of 8% CO₂ (v/v in air).

The acidification of the culture media subjected to the different tested conditions in connection with the cell and tissue viability indexes was assessed every 48 hours for 10 days.

### Staining Protocol

### Cell Cultures—Trypan Blue Staining.

Trypan Blue is a dye that is able to selectively stain dead cells, owing to the extreme selectivity of the cell membrane. Viable cells, having an intact membrane, do not allow this dye to penetrate the cytoplasm; by contrast, Trypan Blue easily enters dead cells, making them distinguishable from the live cells by a rapid analysis under the microscope. Trypan Blue is not able to distinguish apoptotic cells from necrotic cells. The cell suspensions are incubated with 5% Trypan Blue for 5 minutes at room temperature; at completion of the incubation, 10 microliters of such a stained cell solution are taken and deposited into a cell count chamber (e.g. Burker chamber) and carefully observed under a light microscope at successive magnifications of 20×, 40×. Then, the live cells and the dead cells are counted referring to one ml of final volume, repeating the count five times and obtaining the average value resulting from the five determinations. Dead cells must appear intensively colored in blue, live cells must not be blue.

### Biopsies—Hematoxylin-Eosin Staining.

This is the basic staining for the microscopic study of animal tissues and enables an improved morphological study thereof under the light microscope. Thanks to hematoxylin or Mayer's hemallume, it colors in blue the negatively charged cell components, such as nucleic acids, membrane proteins and cell membranes, elastin, which are thus said to be basophilic. Thanks to eosin, it colors in red the positively charged (acidic) components, such as cell proteins in certain (eosinophilic) cells and collagen fibers, which are thus said to be acidophilic.

### Results

### Example 1

### Light Microscopy

In all the controls tested at 48, 96, 192, 240, 288 hours of incubation, with no replacement of culture medium, after Trypan Blue staining for cell cultures and hematoxylin-eosin staining for tissues, a progressive increase in cell mortality ratio and tissue degradation was detected after 192 hours of incubation.

The samples showed a widespread eutrophism with a specific morphology and a viability of between 70% and 100% for each cell type tested, up to the end of incubation (288 hours) and under all the culture conditions as described in Tables 1-bis, 2-bis, 3-bis, 4-bis, 5-bis, 6-bis and 7-bis.

The Sample-biopsies, subjected to the culture conditions as described in Tables 1-bis, 2-bis, 3-bis, 4-bis, 5-bis, 6-bis and 7-bis appeared to be eutrophic and with a substantially similar morphology at Time zero (T zero) up to the end of incubation (288 hours).

The phenol red indicator was used to monitor the condition of the basic culture medium at Time zero. The basic medium was colored in red=normal: pH 7.2-7.4.

The results showed that the use of additional culture conditions, as described in Tables 1-bis, 2-bis, 3-bis, 4-bis, 5-bis, 6-bis and 7-bis, significantly slowed down the acidification of the basic culture media in all the tested samples (at 288 hours the phenol red indicator slightly shifted to light pink, pH 7.0-7.1).

In parallel, the cell mortality ratio and morphological tissue abnormalities did not undergo particular increases up to the end of the experimental incubation (288 hours: 70-72% cell viability ratio and 30-28% cell mortality ratio, average values of all the tested cell samples; slight tissue hypochromia in all the tested biopsy samples with maintenance of the original morphology).

By contrast, all the tested controls showed that the use of the culture medium alone, without anti-acidosis additions, gave a slight acidification of the supernatant beginning from 48 hours (48 hours, the phenol red indicator shifted to light pink, pH 7.0-7.1), with a progressive decrease of the pH (288 hours, the phenol red indicator slightly shifted to lemon yellow, pH 5.08-6.0); in parallel, the cell mortality ratio and morphological tissue abnormalities underwent a continuous increase up to the end of the experimental incubation (288 hours: 4-8% cell viability ratio and 92-96% cell mortality ratio, average values of all the tested cell controls; exfoliation with tissue breakdown in all the tested biopsy controls).

Characterization of Cell Cultures Treated with 1-Bis, 2-Bis, 3-Bis, 4-Bis, 5-Bis, 6-Bis and 7-Bis Versus Untreated Controls

From Time Zero to Time 288 hours the cultures did not undergo exchange of the medium.

The results related to expression of Viability (V)/Mortality (M) were reported by means of a quantitative scale as average percentage values (Trypan Blue Staining), as follows in Tables 1 and 2.

TABLE 1

| Chronogram | | Cell cultures | | | | | | | | | | | | | | | | | |
|---|---|---|---|---|---|---|---|---|---|---|---|---|---|---|---|---|---|---|---|
| Incubations | | Monocyte cell line THP-1 | | | | | | | | | | | | | | | | | |
| Conditions | | Controls | | | | Samples | | | | | | | | | | | | | |
| Viabi- | Morta- | Ctrl-1 | | Ctrl-2 | | 1-bis | | 2-bis | | 3-bis | | 4-bis | | 5-bis | | 6-bis | | 7-bis | |
| lity | lity | V | M | V | M | V | M | V | M | V | M | V | M | V | M | V | M | V | M |
| (V %) | (M %) | % | % | % | % | % | % | % | % | % | % | % | % | % | % | % | % | % | % |

| | | | | | | | | | | | | | | | | | | |
|---|---|---|---|---|---|---|---|---|---|---|---|---|---|---|---|---|---|---|
| Time Zero | 99% | 1% | 99% | 1% | 99% | 1% | 99% | 1% | 99% | 1% | 99% | 1% | 99% | 1% | 99% | 1% | 99% | 1% |
| 12 hours | 98% | 2% | 99% | 1% | 99% | 1% | 99% | 1% | 98% | 2% | 97% | 3% | 98% | 2% | 99% | 1% | 99% | 1% |
| 24 hours | 96% | 4% | 97% | 3% | 98% | 2% | 98% | 2% | 98% | 2% | 98% | 2% | 98% | 2% | 98% | 2% | 98% | 2% |
| 48 hours | 82% | 18% | 80% | 20% | 98% | 2% | 98% | 2% | 98% | 2% | 98% | 2% | 98% | 2% | 98% | 2% | 98% | 2% |
| 96 hours | 70% | 30% | 73% | 27% | 90% | 10% | 92% | 8% | 89% | 11% | 90% | 10% | 90% | 10% | 91% | 9% | 92% | 8% |
| 192 hours | 50% | 50% | 43% | 57% | 80% | 20% | 82% | 18% | 85% | 15% | 85% | 15% | 87% | 13% | 87% | 13% | 89% | 11% |
| 240 hours | 20% | 80% | 13% | 87% | 78% | 22% | 78% | 22% | 71% | 29% | 77% | 23% | 80% | 20% | 84% | 16% | 87% | 13% |
| 288 hours | 8% | 92% | 8% | 92% | 60% | 40% | 65% | 35% | 72% | 28% | 71% | 29% | 72% | 28% | 80% | 20% | 82% | 18% |

Key
Viability = no blue color with Trypan Blue Staining
Mortality = presence of blue color with Trypan Blue Staining
Viability % = average percentage value of live cells per ml
Mortality % = average percentage value of dead cells per ml

TABLE 2

| Chronogram | | Cell cultures | | | | | | | | | | | | | | | | | |
|---|---|---|---|---|---|---|---|---|---|---|---|---|---|---|---|---|---|---|---|
| Incubations | | Canine primary hepatic cells | | | | | | | | | | | | | | | | | |
| Conditions | | Controls | | | | Samples | | | | | | | | | | | | | |
| Viabi- | Morta- | Ctrl-1 | | Ctrl-2 | | 1-bis | | 2-bis | | 3-bis | | 4-bis | | 5-bis | | 6-bis | | 7-bis | |
| lity | lity | V | M | V | M | V | M | V | M | V | M | V | M | V | M | V | M | V | M |
| (V %) | (M %) | % | % | % | % | % | % | % | % | % | % | % | % | % | % | % | % | % | % |

| | | | | | | | | | | | | | | | | | | |
|---|---|---|---|---|---|---|---|---|---|---|---|---|---|---|---|---|---|---|
| Time Zero | 88% | 12% | 88% | 12% | 88% | 12% | 88% | 12% | 88% | 12% | 88% | 12% | 88% | 12% | 88% | 12% | 88% | 12% |
| 12 hours | 78% | 22% | 87% | 13% | 88% | 12% | 87% | 13% | 88% | 12% | 87% | 13% | 89% | 11% | 89% | 11% | 89% | 11% |
| 24 hours | 75% | 25% | 77% | 23% | 90% | 10% | 92% | 8% | 89% | 11% | 88% | 12% | 91% | 9% | 93% | 7% | 92% | 8% |
| 48 hours | 62% | 38% | 60% | 40% | 90% | 10% | 94% | 6% | 93% | 7% | 93% | 7% | 90% | 10% | 92% | 8% | 92% | 8% |
| 96 hours | 40% | 60% | 53% | 47% | 82% | 18% | 85% | 15% | 81% | 19 % | 82% | 18% | 86% | 14% | 90% | 10% | 94% | 6% |
| 192 hours | 32% | 68% | 40% | 60% | 77% | 23% | 78% | 22% | 80% | 20% | 80% | 20% | 82% | 22% | 88% | 12% | 89% | 11% |
| 240 hours | 18% | 82% | 23% | 77% | 72% | 28% | 72% | 28% | 74% | 26% | 73% | 27% | 77% | 23% | 83% | 27% | 83% | 17% |
| 288 hours | 2% | 96% | 6% | 96% | 62% | 38% | 69% | 31% | 70% | 30% | 70% | 30% | 74% | 26% | 77% | 23 % | 78% | 22% |

Key
Viability = no blue color with Trypan Blue Staining
Mortality = presence of blue color with Trypan Blue Staining
Viability % = average percentage value of live cells per ml
Mortality % = average percentage value of dead cells per ml

### Example 2

### In Vivo

### Rescue Anti-Acidosis Therapy.

### Explanations:

The acid-base balance is regulated by the respiratory system and the kidneys.

Just a few minutes are sufficient for a patient to ventilate more or less rapidly, causing a decrease or increase in PCO₂ and, thereby, a change in pH, towards the low or high end of the range, respectively. Instead, kidneys require 24-48 hours to absorb or eliminate the bicarbonate ion, causing also an increase or decrease in pH, respectively, in order to compensate for the acidosis or alkalosis condition. Change of pH towards the low end is known as acidosis (below 7.35), change of pH towards the high end is known as alkalosis (above 7.45). Acidosis and alkalosis may be respiratory or metabolic, depending on the mechanism they rely on. Metabolic acidosis in small animals, and in mammals in general, is caused by:
excess acid production (diabetic ketoacidosis, exercise: lactic acidosis);
bicarbonate ion loss (diarrhea, renal failure and failure by insufficient renal bicarbonate resorption).

The diet of cats and dogs is often deficient in enzymes, which are in non-heat-treated food. In the absence of these enzymes, the digestive and absorption processes are stressful for pancreas, liver and intestine, and cause excess waste that overloads the kidneys. Urine pH in carnivores must be acidic, with a pH value between 5.5-6.0. A higher urine acidity may derive from feverish states, prolonged fasting, diabetes mellitus, or metabolic and respiratory acidosis. With time, the overburdened organs become functionally insufficient and favor build-up of catabolites and acids, predisposing to the onset of serious acidosis conditions and consequences thereof. In animals in general and mammals, anaerobiosis (lack of oxygen) and tissue acidosis constitute two factors that favor carcinogenesis.

Blood acidosis can create an electrostatic force around the cell membrane which induces piling up of red cells in the capillaries, preventing the correct circulation thereof. The scarcely wetted tissues do not allow the immune cells to intervene properly in the removal of degenerated cells.

### Study Under Compassionate-Regime

The rescue anti-acidosis therapy was administered in two human cases. The following were treated:
two human (male) cases affected by decompensated insuline-dependent diabetes mellitus (IDDM).

In order to estimate the anti-acidosis tolerability and therapeutic effectiveness, the following were treated under compassionate regime:
twenty-eight animals (twelve dogs and twelve cats different in breed and size, and four sheep) which exhibited serious acidosis conditions (metabolic acidosis, lactic acidosis and complications thereof) attributable to different disease outcomes (for sheep, copper food poisoning; for all: chronic diarrhea, increased production of fixed acids such as lactic acid and keto-acids, hypoadrenocorticism, decompensated diabetes, pharmacological toxicity, neoplasias, seizures, hepatic encephalopathy, hepatopathies, thyroiditis, infectious diseases, acute post-traumatic metabolic reactions, calcium dysmetabolisms, glycol ethylene or salicylate poisoning, general metabolic toxicity conditions, general dysmetabolic conditions, chronic renal failure, electrolyte imbalances, general cardiopathies and myopathies, dyspnea, administration of carbonic anhydrase inhibitors). At the enrolment examination, the selected subjects exhibited the above-indicated conditions.

### Monitoring

Hematochemical tests: lactacidemia, hemochrome+leukocyte formula, glycemia, creatininemia, azotemia, amylasemia, LDH, CK, chloremia, blood potassium, blood sodium, blood magnesium, calcemia, venous EGA, complete urine test. Instrumental tests: ECG, chest x-ray. Clinical check-ups occurred twice a week until the disease was resolved.

### Man

### Metabolic Acidosis and Lactic Acidosis.

Significant hyperlactacidemia in both cases (lactacidemia normal value<2.5 mmol/l; case 1, lactacidemia 5 mmol/l; case 2: lactacidemia 9 mmol/l).

At the end of the first therapeutic round (oral administration for seven days) all the pathological parameters returned within the standard physiological ranges (lactacidemia in case 1: 1.3 mmol/l and in case 2: 2.0 mmol/l; lactacidemia nv<2.5 mmol/l).

### Dogs

### Post-Seizure Metabolic Acidosis Conditions.

At the end of the first therapeutic round (oral administration for seven days) all the pathological parameters returned within the standard physiological ranges.

### Cats

### Metabolic Acidosis Conditions Following Chronic Renal Failures.

At the end of the first two therapeutic rounds (oral administration for fourteen days at relevant concentrations) all the pathological parameters returned within the standard physiological ranges.

### Sheep

Sheep with copper poisoning were treated. At the end of the first therapeutic round (oral administration for seven days), in conjunction with zinc administration to chelate the excess copper, all the pathological parameters returned within the standard physiological ranges.

### REFERENCES

1. Muratore O et al. Evaluation of the trophic effect of human placental polydeoxyribonucleotide on human knee skin fibroblasts in primary culture. Cell Mol Life Sci. 1997 March; 53(3):279-85.
2. Wu S et al. Surrogate splicing for functional analysis of sesquiterpene synthase genes. Plant Physiol. 2005 July; 138(3):1322-33.
3. Galeano M et al. Polydeoxyribonucleotide stimulates angiogenesis and wound healing in the genetically diabetic mouse. Wound Repair Regen. 2008 March-April; 16(2):208-17.
4. Cetingil A I et al. Heparin-like anticoagulant occurring in association with chronic nephritis. Br Med J. 1959 Jul. 11; 2(5140):38-9.
5. Stella C C et al. Defibrotide in combination with granulocyte colony-stimulating factor significantly enhances the mobilization of primitive and committed peripheral blood progenitor cells in mice. Cancer Res. 2002 Nov. 1; 62(21):6152-7.
6. Paul W et al. The effect of defibrotide on thromboembolism in the pulmonary vasculature of mice and rabbits and in the cerebral vasculature of rabbits. Br J. Pharmacol. 1993 December; 110(4):1565-71.
7. Richardson P G et al. Treatment of severe veno-occlusive disease with defibrotide: compassionate use results in response without significant toxicity in a high-risk population. Blood. 1998 Aug. 1; 92(3):737-44.
8. Guizzardi S et al. Polydeoxyribonucleotide (PDRN) promotes human osteoblast proliferation: a new proposal for bone tissue repair. Life Sci. 2003 Aug. 29; 73(15):1973-83.
9. Coe F L et al. Evidence for secondary hyperparathyroidism in idiopathic hypercalciuria. J Clin Invest. 1973 January; 52(1):134-42.
10. Krieger N S et al. Acidosis inhibits osteoblastic and stimulates osteoclastic activity in vitro. Am J. Physiol. 1992 March; 262(3 Pt 2):F442-8.
11. Bushinsky D A et al. Chronic acidosis-induced alteration in bone bicarbonate and phosphate. Am J Physiol Renal Physiol. 2003 September; 285(3):F532-9.

1-22. (canceled)

23. A composition comprising polydeoxyribonucleotides (PDRNs) and at least a carnitine compound selected from the group consisting of carnitine, a carnitine ester acylcarnitine, levorotatory forms thereof and simple or complexed derivatives thereof.

24. The composition of claim 23, wherein the polydeoxyribonucleotides (PDRNs) comprise a mixture of non-coding deoxyribonucleotide chains having a molecular weight distribution comprised between about 20 kDalton and about 2500 kDalton.

25. The composition of claim 24, wherein the polydeoxyribonucleotides derives from an animal source.

26. The composition of claim 23, when in a liquid form comprises the polydeoxyribonucleotides (PDRNs) at a concentration of about 0.001 to about 50 mg/ml and when in a solid form comprises the polydeoxyribonucleotides (PDRN) at a concentration of about 5 to about 500 mg/g.

27. The composition of claim 23, when in a liquid form comprises the carnitine compound at a concentration of about 5 to about 3000 mg/l and when in a solid form comprises the carnitine compound at a concentration of about 100 to about 5000 mg/g.

28. The composition of claim 24, further comprising a calcium salt.

29. The composition of claim 28, wherein the calcium salt is selected from the group consisting of calcium carbonate, calcium gluconate, calcium lactate, calcium phosphate, dibasic calcium phosphate, calcium nitrate, calcium citrate, monocalcium citrate, tricalcium citrate, calcium dicitrate, and tricalcium dicitrate tetrahydrate.

30. The composition of claim 28, when in a liquid form comprises the calcium salt at a concentration of calcium ions of about 1 to about 40 mM, and when in a solid form comprises the calcium salt at a concentration of calcium ions of about 50 to about 2000 mg/g.

31. The composition of claim 24 comprising at least one vitamin.

32. The composition of claim 31 wherein said vitamin is selected from the group consisting of: a vitamin from the B group, a vitamin from the D group, a derivative vitamin of the B group, a derivative vitamin of the D group and compositions thereof.

33. The composition of claim 32, wherein the vitamin of the D group is selected from the group consisting of calciferol, ergocalciferol, lumisterol, cholecalciferol, dihydroergocalciferol, sitocalciferol, calcitriol and calcifediol, and the vitamin of the B group is selected from thiamine (vitamin B1), riboflavin (vitamin B2), niacin, nicotinic acid, nicotinamide (vitamin B3), adenine (vitamin B4 or vitamin-like factor), pantothenic acid, pantenol, pantenine (vitamin B5), pyridoxamine, pyridoxine and pyridoxal (vitamin B6).

34. The composition of claim 32, when in a liquid form comprises the vitamin of the D group at a concentration of about 0.1 to about 10.0 μg/l, and the vitamin of the B group at a concentration of about 1 to about 1000 mg/l, and when in a solid form comprises the vitamin of the D group at a concentration of about 0.1 to about 10.0 μg/g.

35. The composition of claim 24, comprising a salt selected from the group consisting of potassium and sodium.

36. The composition of claim 35, wherein the salt is selected from the group consisting of sodium citrate, potassium citrate, monosodium citrate, monopotassium citrate, disodium citrate, trisodium citrate, tripotassium citrate, sodium carbonate, potassium carbonate, sodium bicarbonate, potassium bicarbonate, sodium sulfate, potassium sulfate, sodium bisulfate, potassium bisulfate, sodium chloride, potassium chloride, sodium tartrate, potassium tartrate, sodium phosphate, potassium phosphate, disodium inosinate, dipotassium inosinate, sodium ascorbate, potassium ascorbate, sodium nitrate, potassium nitrate, or derivatives thereof.

37. The composition of claim 35, when in a liquid form comprises the salt at a concentration of about 1 to about 1000 mg/l and when in a solid form comprises the salt at a concentration of about 5 to about 7000 mg/g.

38. The composition of claim 24, in the form of a pharmaceutical composition, a medical device or a dietary supplement.

39. The composition of claim 38, in a formulation suitable for parenteral or enteral administration.

40. The composition of claim 39, wherein said formulation is suitable for administration selected from the group consisting of: intravenous, intramuscular, infusional, oral, sublingual and rectal.

41. The composition of claim 39, in a form selected from the group consisting of: an oral solution, oral gel, tablet, capsule, effervescent tablet, coated tablet, powder, granulate, suppository, injectable solution and suspension.

42. A method for treating lactic acidosis or metabolic acidosis, comprising administering to a subject in need thereof a therapeutically effective amount of the composition of claim 23.
